(19) United States
(12) Patent Application Publication
(10) Pub. No.: US 2025/0205376 A1
(43) Pub. Date: Jun. 26, 2025

(54) MICROFLUIDIC FABRICATION OF INFUSED SILICA MICROSPHERES

(71) Applicants: Lewis I. Larsen, Albuquerque, NM (US); Nichlaus J. Carroll, Albuquerque, NM (US); Reed G. Selwyn, Albuquerque, NM (US); Gabriel P. Lopez, Penasco, NM (US); Andrew P. Shreve, Santa Fe, NM (US)

(72) Inventors: Lewis I. Larsen, Albuquerque, NM (US); Nichlaus J. Carroll, Albuquerque, NM (US); Reed G. Selwyn, Albuquerque, NM (US); Gabriel P. Lopez, Penasco, NM (US); Andrew P. Shreve, Santa Fe, NM (US)

(21) Appl. No.: 18/990,943

(22) Filed: Dec. 20, 2024

Related U.S. Application Data
(60) Provisional application No. 63/614,101, filed on Dec. 22, 2023.

(51) Int. Cl. A61K 51/12

(52) U.S. Cl. CPC A61K 51/1255

(57) ABSTRACT

The disclosure provides a composition comprising a monodispersion of silica glass microspheres infused with a positron emission tomography (PET) radiotracer and a method of making the same. The composition can be used as a positron emission tomography (PET) imaging surrogate for predicting in vivo behavior of therapeutic Y-90 microspheres. Compositions and methods are also described utilizing a therapeutic nuclide. In various examples, the monodispersion of silica glass microspheres can be infused with copper, gallium, or fluorine.

### CROSS-REFERENCE TO RELATED APPLICATION

This application claims the benefit of priority to U.S. Provisional Patent Application Ser. No. 63/614,101, filed Dec. 22, 2023, the disclosure of which is incorporated herein in its entirety by reference.

### BACKGROUND

Liver cancer is the second deadliest cancer worldwide, after lung cancer, with over 800,000 people succumbing to it per year. Hepatocellular carcinoma (HCC) is the primary malignancy of the liver and accounts for roughly 90% of all liver cancer cases. While liver transplant or tumor resection are potentially curative, they are often not viable options due to organ supply issues and/or risk of overwhelming damage to the liver during surgery. One increasingly used form of treatment for unresectable HCC is selective internal radiation therapy (SIRT), which works by injecting radioactive Y-90 microspheres via the hepatic artery. The hepatic artery provides over 80% of the blood flow to HCC tumors, while normal liver parenchyma is mostly supplied by the portal vein. This makes for favorable odds when injecting into the hepatic artery, the therapeutic will mostly be delivered to tumorigenic tissue instead of healthy liver tissue. Y-90 emits pure beta radiation and has had clinical relevance for intravascular tumor control for many decades. The high-energy beta particles penetrate 2.5 to 10 mm into soft tissue, which is suitable distance range for tumor eradication. The Y-90 microspheres are roughly 20-30 μm in diameter, making them small enough to easily flow through the hepatic artery but large enough to not pass through the capillary bed of the liver. However, such processes can involve leaching radioactive material from the microsphere or shunting microsphere into unwanted parts of the body, such as the lungs. Despite precautions taken to prevent lung shunting via developments of microsphere morphology, the issue sometimes remains depending on candidate internal anatomy.

Not every liver tumor is suitable for SIRT due to nuances in individual tumor biology. This may cause many Y-90 microspheres to accumulate outside of the inner tumor vasculature and damage non-tumorigenic tissue. Therefore, efforts have been made to predict the in vivo behavior of Y-90 microspheres (tumor-to-liver ratio or TLR) by injecting a radioactive surrogate prior to Y-90 injection. Tc-99m macroaggregated albumin (MAA) particles have been used as pre-treatment radiotracers to predict microsphere distribution with varying levels of success. Tc-99m MAA are imaged via single photon emission computed tomography (SPECT). Unfortunately, these particles have a host of issues which limit their utility as Y-90 microsphere surrogates. Biodegradable and possibly immunogenic MAA particles are not morphologically representative of Y-90 glass microspheres and as a result their in vivo behavior may not always be sufficiently representative of Y-90 glass microspheres. Tc-99m MAA particle size can vary between 10 μm and 150 μm, which is quite a large distribution when compared to the monodisperse Y-90 glass microspheres. Larger MAA particles may embolize small blood vessels outside the tumor while small particles may end up in the lungs. MAA particles thus can display an overly high lung shunting factor and can lead to underestimation for Y-90 dose selection for conserving health of normal tissue and lungs. Shunting inaccuracies can also disqualify HCC lesions which otherwise would have responded positively to SIRT.

There remains a need for improved pre-treatment radiotracers, SIRT microsphere surrogates, and methods of predicting in vivo behavior of Y-90 microspheres.

### SUMMARY OF THE INVENTION

The disclosure provides a composition comprising a monodispersion of silica glass microspheres infused with a positron-emission tomography (PET) radiotracer or therapeutic nuclide.

The disclosure also provides a method of preparing a monodispersion of silica glass microspheres infused with a positron-emission tomography (PET) radiotracer or therapeutic nuclide, comprising, preparing an aqueous sol of infused silica precursors from a silicate precursor, a radiotracer or therapeutic nuclide precursor, and an aqueous solvent; preparing microfluidic droplets from an emulsification of the aqueous sol and an organic oil continuous fluid dispersed by a microfluidic device; and providing a two-step thermal treatment, which includes a first thermal step that heats the microfluidic droplets sufficiently to polymerize the droplets by solvent evaporation, and a second thermal step that heats the polymerized droplets sufficiently to consolidate an infusion-silica matrix and evaporate residual components.

The disclosure yet further provides a method of predicting in vivo behavior of therapeutic Y-90 microspheres comprising administering to subject a composition comprising a monodispersion of silica glass microspheres infused with a positron-emission tomography (PET) radiotracer.

The present disclosure provides a composition that can be useful as a pre-treatment radiotracers or Y-90 microspheres surrogate for predicting the in vivo behavior of therapeutic Y-90 microspheres with the advantages of positron-emission tomography (PET). In various aspects, one advantage of the presently described composition and methods is that they permit access to improved surrogates and use of PET radiotracers for quantitative assessment and pretreatment dosimetry. Positron-emission tomography (PET) can advantageously provide improved image and spatial resolution relative to the SPECT imaging that is used with Tc-99m MAA for surrogate modeling of Y-90. Positron-emission tomography can also have advantages of improved accuracy of pretreatment dose distribution maps and shorter imaging times.

In various further aspects, the presently described composition can provide one or more of the advantages of (i) a quickly and conveniently fabricated composition; (ii) a tunable composition; (iii) advantages with respect to point-of-care production and personalized fabrication; (iv) improved stability and minimization of leaching relative to surface-bound radiotracers; (v) flexible fabrication utilizing bottom up synthesis and top-down microfluidics; and (vi) a generalized approach suitable for infusion of a diverse set of radiotracers.

### BRIEF DESCRIPTION OF THE FIGURES

The drawings illustrate generally, by way of example, but not by way of limitation, various aspects of the present invention.

FIG. 1 provides a schematic depicting the microfluidic and sol-gel chemistry fabrication approach for silica glass microparticles doped with copper, gallium, or fluorine, a process which can be completable in as little as two hours for a sample of 500,000 doped silica glass microspheres.

FIG. 2A provides an optical microscope image of 80.5±5.4 μm (CV of 6.7%) copper nitrate-infused sol precursor droplets stabilized in ABIL/hexadecane continuous fluid.

FIG. 2B provides a SEM micrograph of copper-infused silica particles after thermal treatment.

FIG. 2C provides a SEM micrograph of fluorine-infused silica particles after thermal treatment.

FIG. 2D provides histogram size distributions for copper, gallium, and fluorine-infused silica particles with a relatively narrow size distribution.

FIG. 3A provides a chart illustrating the effect of increasing infusion precursor molar amounts on resulting silica particle infusion mole percentage, showing an increase in infusion percentage with increasing initial molar amounts of infusion precursor.

FIG. 3B provides a chart illustrating the fraction of initial copper, gallium, and fluorine infusion added to precursor that is retained in the silica particles post-furnace treatment, showing that fractions generally decrease with increasing initial precursor molar amounts.

FIG. 4A provides a SEM micrograph showing cross sectioned copper-infused microspheres, showing that small spherical copper/copper oxide clusters are observed dispersed within the silica matrix.

FIG. 4B provides a SEM micrograph showing cross sectioned gallium-infused microspheres, showing that gallium appears more homogeneously distributed throughout the silica particles.

FIG. 5 provides SEM images of Ga-68 microspheres one-week post-fabrication.

FIG. 6 provides a plot depicting the natural decay of Ga-68 over the span of the 24 minutes of ten saline rinse cycles. The plot also provides predicted activity (expected activity based on Ga-68 half-life) alongside observed activity as measured by dose calibrator showing that the measured activity matches the expected natural decay.

FIG. 7 provides a schematic of flow-focusing two-channel microfluidic device.

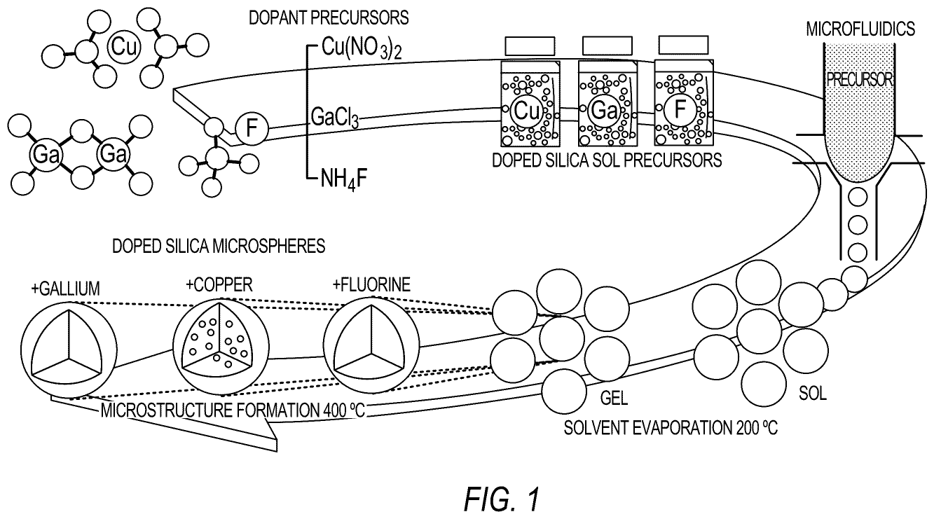

### DETAILED DESCRIPTION OF THE INVENTION

Reference will now be made in detail to certain embodiments of the disclosed subject matter, which may be illustrated in part via the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. The term “about” as used herein can allow for a degree of variability in a value or range, for example, within 10%, within 5%, or within 1% of a stated value or of a stated limit of a range, and includes the exact stated value or range.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Various aspects of the present disclosure can relate to a “monodispersion” or a “monodisperse composition”. Those skilled in the art understand that chemical compositions, particularly colloidal systems, can be described in terms of their constituent particles, and those skilled artisans will readily recognize the distinction between monodisperse systems and polydisperse systems. Generally, polydisperse systems exhibit a wider variation of particle size, while monodisperse systems exhibit narrower variation with respect to particle size. Without intending to limit any aspect or feature of the present disclosure, monodispersions exhibit a high degree of uniformity relative to polydisperse systems, which can offer functional advantages with respect to exhibited physical and chemical properties, as well as reproducibility and precision in providing the same. It can be useful to describe a monodispersion in terms of its coefficient of variation, as doing so can provide a quantitative description of how particles vary. Various related terms are envisaged as encompassed by the present disclosure, including systems that are quasi-monodisperse, nearly monodisperse, moderately monodisperse, highly monodisperse, other than quasi-monodisperse, a system in which particle sizes are controlled toward a specific range of one or more particle size bands or particle size target values, a system in which control of a specific range of one or more particle size bands or particle size target value is a achieved, or any combination of such descriptions. In various aspects, the monodispersion of the present disclosure can be specified by using one or more of the coefficient of variation values and ranges described, including combinations of such values as upper and lower bounds, to further specify a particular degree of uniformity.

Various aspects of the present invention can be better understood by reference to the following Examples which are offered by way of illustration. The present invention is not limited to the Examples given herein.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Monodisperse Infused Silica Glass Microspheres

The disclosure provides a composition comprising a monodispersion of silica glass microspheres infused with a positron-emission tomography (PET) radiotracer. The composition can be useful for predicting in vivo behavior of therapeutic Y-90 spheres.

Silica glass microspheres are microscale particles having a spherical morphology and a silica matrix. The silica matrix is generally comprised of a network of Si—O bonds. As described herein, silica glass microspheres can be infused with one or more positron-emission tomography (PET) radiotracer. The resulting infused silica glass microsphere incorporates the radiotracer within the bulk of the microsphere body, rather than labeled on the particle surface. The infused radiotracer can be incorporated or entrapped within the silica matrix. Conventional use of radiotracers typically involves chemically tethering them to the surface of particles or chelating. Radiotracers that are merely chelated, surface-coated, or otherwise labeled onto the particle surface can suffer from leaching radioactivity and other problems. In contrast, radiotracers that are incorporated via infusion into the bulk of the microsphere body obviates the problem of leaching radioactivity and other surface-mediated problems. The presently described microspheres can thus more closely represent the morphology of Y-90 SIRT microspheres. They are also resistant to degradation relative to particles that utilize surface-attached radiotracers or radiotracers bound merely by pendant organic moieties. As such, the presently described microspheres can closely replicate actual behavior of clinically used Y-90 resin or glass microspheres.

The composition comprises silica glass microspheres having a focused distribution of particle sizes so as to represent a monodispersion. For example, the composition can have a monodispersion of silica glass microspheres wherein about or at least 50%, 60%, 70%, 80%, 90%, 95%, or 99% of silica glass microspheres in the composition are particles having a diameter of 10 μm to 50 μm. The composition can have a monodispersion of silica glass microspheres wherein about or at least 90%, 95%, or 99% of silica glass microspheres in the composition are particles having a diameter of 10 μm to 30 μm. The monodispersion can have a coefficient of variation (CV) of less than or about 20%, 19%, 18%, 17%, 16%, 15%, 14%, 13%, 12%, 11%, 10%, 9%, 8%, 7%, 6%, 5%, 4%, 3%, 2%, or 1%. As another example, the monodispersion can have a coefficient of variation of 10% to 15%, 5% to 15%, 1% to 15%, 5% to 10%, or 1% to 10%. To avoid ambiguity, this document refers to the coefficient of variation in terms of percent unless otherwise indicated. In various aspects, it can be useful to describe the present compositions in terms of its monodispersion of precursor components or pre-processed microfluidic droplets, which may be described with a first coefficient of variation. For example, the monodispersion of precursor components or pre-processed microfluidic droplets can have a coefficient of variation of less than 15%, 14%, 13%, 12%, 11%, 10%, 9%, 8%, 7%, 6%, 5%, 4%, 3%, 2%, or 1%, or about 5% to about 7%, about 1% to about 7%, about 1% to about 10%, about 5% to about 15%, 5% to about 10%, or 1% to about 15%. It can also be useful to describe the present compositions in terms of the monodispersion of its resulting infused silica glass microsphere product, which can be described with a second coefficient of variation. For example, the post-processed infused silica glass microspheres can have a coefficient of variation of less than or about 20%, 19%, 18%, 17%, 16%, 15%, 14%, 13%, 12%, 11%, 10%, 9%, 8%, 7%, 6%, 5%, 4%, 3%, 2%, or 1%, or about 10% to about 15%, about 5% to about 15%, about 1% to about 15%, about 5% to about 10%, or about 1% to about 10%. In various aspects, the processing stage can cause particle size to shrink and coefficient of variation to increase, which is one reason why it may be useful to discuss separate ranges of coefficient of variation depending on context. In a further example, the pre-processed microfluidic droplets can have a monodispersion with a coefficient of variation of less than 7% or about 5% to about 7%, and the post-processed infused silica glass microspheres can have a monodispersion with a coefficient of variation of less than 15%, about 5% to about 15%, about 7% to about 15%, about 9% to about 15%, or about 10% to about 15%.

In various aspects, the composition can comprise silica glass microspheres having a diameter of 10 μm to 100 μm. In further aspects, the composition can comprise silica glass microspheres having a diameter of 10 μm to 50 μm. In yet further examples, the composition can comprise silica glass microspheres having a diameter of 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, or 50 μm. In further aspects, the silica glass microspheres have a diameter of 10 μm to 40 μm, 10 μm to 30 μm, 20 μm to 40 μm, or 20 μm to 30 μm.

Positron-emission tomography (PET) radiotracers are radioactive isotopes that emit positrons. In positron-emission tomography, a radioisotope is injected into a subject's body. The radioisotope decays and emits a positron, which interacts with an ordinary electron to release gamma rays. These gamma rays are detected by the device to generate an image. Positron-emission tomography (PET) radiotracers can be used to understand and anatomically map tissue and organ systems in a subject. They can also be used to image and measure blood flow, oxygen usage, tissue pH, neurotransmitter activity, and the proliferation of cancer cells, among other physiological and biochemical processes.

Examples of positron-emission tomography (PET) radiotracers include Cu-64, Ga-68, Zr-89, and F-18. Cu-64 has a convenient 12.7-hour half-life and enables imaging up to 48 hours post-injection. F-18 is most commonly administered by conjugating to a bioactive molecule analog such as fluorodeoxyglucose (FDG). FDG can itself to be useful in diagnosis of carcinoma of unknown primary by whole body PET imaging, with successful detection rate of between 33% and 57%. Ga-68 can also be advantageously used for imaging HCC. Ga-68 can be advantageously produced in-house from commercially available germanium-68/gallium-68 generators as opposed to other isotopes (which often require an off-site cyclotron or nuclear reactor). Inconvenient infrastructure used to produce radioisotopes and chemical reactions for radiotracer fabrication is a barrier which can impede the use of PET. Ga-68 is thus a particularly advantageous PET radioisotope given the availability of germanium-68/gallium-68 generators to overcome logistical transit issues. Ga-68 has been used as a radionuclide since the 1960s, typically uses have relied on chelated onto DOTATATE, DOTATOC, and other chelate groups. However, radiotracers that are merely chelated, surface-coated, or otherwise labeled onto the particle surface can suffer from leaching radioactivity and other problems.

In various aspects, the composition is formulated for administration to a subject. The composition can comprise one or more pharmaceutically acceptable carriers. The composition can be administered to image tissue or organ systems in a subject. The composition can be administered as a pre-treatment radiotracer to predict distribution of radioactive Y-90 microspheres.

The monodisperse silica microspheres can contain from 0.1 mol % to 5 mol % of a positron-emission tomography (PET) radiotracer. For example, the microspheres can contain 0.1 mol %, 0.2 mol %, 0.3 mol %, 0.4 mol %, 0.5 mol %, 0.6 mol %, 0.7 mol %, 0.8 mol %, 0.9 mol %, 1 mol %, 2 mol %, 3 mol %, 4 mol %, or 5 mol % of a positron-emission tomography (PET) radiotracer. As a further example, the microspheres can contain up to 5 mol % copper or gallium. As another example, the microspheres can contain up to 2 mol % fluorine.

### Fabrication of Monodisperse Infused Silica Glass Microspheres

The disclosure provides a method of preparing a monodispersion of silica glass microspheres infused with a positron-emission tomography (PET) radiotracer, comprising: preparing an aqueous sol of infused silica precursors from a silicate precursor, a radiotracer precursor, and an aqueous solvent; preparing microfluidic droplets from an emulsification of the aqueous sol and an organic oil continuous fluid dispersed by a microfluidic device; and providing a two-step thermal treatment, which includes a first thermal step that heats the microfluidic droplets sufficiently to polymerize the droplets by solvent evaporation, and a second thermal step that heats the polymerized droplets sufficiently to consolidate a infusion-silica matrix and evaporate residual components.

The radiotracer precursor is incorporated when preparing the aqueous sol of silica precursors. The aqueous sol is prepared from a silicate precursor, a radiotracer precursor, and an aqueous solvent.

The silicate precursor is a reactive, hydrolysable precursor suitable for providing a silica matrix via a sol-gel process. The specific structure of the silicate precursor is not particularly limited. In various aspects, the silicate precursor can have the structure SiX₄, where X is a hydrolysable group, such as an alkoxy group. For example, the silicate precursor can be an alkyl silicate. The silicate precursor can be a tetraalkyl orthosilicate. The silicate precursor can have the structure SiOR₄, where R is an alkyl group. In various aspects, the silicate precursor is dissolved in an alcohol solvent.

The radiotracer precursor is a compound that provides a source of radiotracer to the sol-gel mixture. The radiotracer precursor typically includes a solvent. The radiotracer precursor is typically a salt, for which one of the counterions is a radionuclide. For example, the radiotracer precursor can be NH₄F, GaCl₃, or Cu(NO₃)₂. The particular salts and sources of radiotracer are not particularly limited. In some aspects, the radiotracer precursor is not a salt. In some aspects, the radiotracer precursor is the radiotracer in a solvent. In some aspects, the radiotracer precursor is in an aqueous solvent, e.g., DI water.

Solvent refers to a liquid that can dissolve a solid, liquid, or gas. Non-limiting examples of solvents including water, alcohols (e.g., ethanol), protic solvents, aprotic solvents, aqueous solvent mixtures, organic solvents, halogenated organic solvents, liquid hydrocarbons including alkane-based solvents, ionic liquids, and combinations thereof. The aqueous sol involves an aqueous solvent, which can be a mixture of water and alcohol.

Microfluidic droplets are prepared by first producing an emulsification of the aqueous sol with an organic oil continuous fluid and then by dispersing it into droplets by use of a microfluidic device.

The organic oil continuous fluid is used to provide a controlled emulsion together with the aqueous sol by using a microfluidic device so as to tune droplet size and, thus, resulting microsphere size. The organic oil continuous fluid can be prepared by dissolving a surfactant in an organic solvent. The surfactant can be a copolyol, such as a silicone-based non-ionic surfactant. The organic solvent is immiscible in the aqueous sol so as to permit emulsion formation. Example organic solvents include aprotic solvents and hydrocarbon solvents, for example one or more of octane, nonane, decane, undecane, dodecane, tridecane, tetradecane, pentadecane, hexadecane, heptadecane, octadecane, which can include linear forms, branched forms, or mixtures thereof. In various aspects, the organic oil continuous fluid comprises a C10-C20 hydrocarbon solvent. In various aspects, the organic oil continuous fluid comprises a copolyol surfactant. The organic oil continuous fluid can comprise hexadecane.

Dispersion of the microfluidic droplets is configured to provide droplets that will subsequently polymerize and calcine to provide microspheres having the target size of 10 μm to 50 μm. Desired droplets sizes can be achieved by controlling volumetric flow rate of the dispersed sol and by controlling volumetric flow rate of the continuous oil. Dispersion at this step can be used to control the resulting size and distribution of the fabricated microspheres. In various aspects, the dispersion is configured to provide droplets having an average particle size of 60 μm to 100 μm. In further aspects, the dispersion is configured to provide droplets having an average particle size of 75 μm to 85 μm. The dispersion can be configured for tight control of droplet sizes so as to provide a monodispersion. For example, the dispersion process can be configured to provide microfluidic droplets having a coefficient of variation of less than or about 20%, 15%, 14%, 13%, 12%, 11%, 10%, 9%, 8%, 7%, 6%, 5%, 4%, 3%, 2%, or 1%. In various further aspects, the dispersion process can be configured to provide microfluidic droplets having a coefficient of variation of about 5% to about 7%, about 1% to about 7%, about 1% to about 10%, about 5% to about 15%, 5% to about 10%, or 1% to about 15%.

The two-step thermal treatment includes a first thermal step and a second thermal step. The first thermal step involves heating the microfluidic droplets sufficiently to polymerize the droplets by solvent evaporation. The second thermal step involves heating the polymerized droplets sufficiently for calcination. During calcination, the infused silica precursors are consolidated to provide the infused-silica matrix. Also during this step, any remaining residual solvents, volatiles, and organics are removed.

In various aspects, the process can involve a single-pot synthesis comprising a modified sol precursor infused with an amount of copper nitrate, gallium chloride, or ammonium fluoride to provide a monodisperse silica microspheres containing up to 5 mol % copper or gallium, and up to 2 mol % fluorine. In various aspects, the monodisperse silica microspheres contain at least 1 mol % of copper, gallium, or fluorine.

In alternative aspects, the presently described composition includes a nuclide selected from copper, gallium, or fluorine in place of the positron-emission tomography (PET) radiotracer. The nuclide can be a radionuclide or a non-radiologic nuclide. The nuclide can be bioactive. The nuclide can serve as an active agent with respect to therapeutic or diagnostic purposes. Some such compounds can be biologically active and therapeutically useful in both radiologic or non-radiologic forms. Where such compositions may benefit from some amount of leaching, for example when they are intended as bioactive therapeutics, higher levels of loading may be suitable. For example, in such alternative aspect, the monodisperse silica microspheres may contain up to or greater than 5 mol % copper or gallium, or up to or greater than 2 mol % fluorine, and at least 1 mol % of copper, gallium, or fluorine.

In a further alternative aspect, the presently described fabrication method can utilize a therapeutic nuclide in place of the radiotracer. Such aspect is analogous to fabrication of radiotracer-infused silica glass microspheres, except instead of infusing a radiologic nuclide, a non-radiologic nuclide is used. Thus, the disclosure also provides a method of preparing a monodispersion of silica glass microspheres infused with a therapeutic nuclide, comprising: preparing an aqueous sol of infused silica precursors from a silicate precursor, a therapeutic nuclide precursor, and an aqueous solvent; preparing microfluidic droplets from an emulsification of the aqueous sol and an organic oil continuous fluid dispersed by a microfluidic device; and providing a two-step thermal treatment, which includes a first thermal step that heats the microfluidic droplets sufficiently to polymerize the droplets by solvent evaporation, and a second thermal step that heats the polymerized droplets sufficiently to consolidate a infusion-silica matrix and evaporate residual components.

The presently described fabrication method can be used for point-of-care fabrication on as needed basis. In various aspects, the entire fabrication method—from production of the aqueous sol to final monodisperse silica glass microspheres—can be completed in several hours. For example, the entire fabrication method can be completed in 1 to 4 hours. In various aspects, the entire fabrication method is completed in less than 2 hours. For example, the entire fabrication method can be completed in about or less than 1 hour, 1.5 hours, 2 hours, 2.5 hours, 3 hours, 3.5 hours, or 4 hours. The speed at which fabrication can be perfomed represents an opportunity for on demand fabrication on a per patient basis.

A typical pre-treatment radiotracing dose for a patient can be, for example, about 100,000 to about 1,000,000 infused silica glass microspheres. This amount of microspheres can be fabricated on site and as needed. The availability of these agents can permit not only radiotracing for predicting in vivo behavior of Y-90 microspheres, but it also permits access to fabricating infused silica glass microspheres that are personalized to a patients needs. For example, the microspheres can even be customized to the condition, ailment, or physiology of the patient. For example, while a 25 μm, 30 μm, or 35 μm size may be typically useful for patients so as to avoid shunting, some patients may be especially vulnerable and benefit from larger particle sizes (e.g., 60 μm, 070 μm, 75 μm, or 80 μm. Use of various sized microspheres at the pre-treatment stage can be useful for identifying optimal treatment and microsphere vehicles for a given patient.

### Experimental Examples

### Aqueous Sol Preparation

Copper and gallium silica sol was prepared by first dissolving up to 50 mg each of Cu(NO₃)₂ or GaCl₃ in 0.75 g of DI water. Next, 0.25 g of tetraethyl orthosilicate (TEOS, Purum>98%) and 1 g of ethanol (EtOH, 99.7%) were added to the mixture under vigorous stirring at room temperature for about 1 minute to hydrolyze and dissolve the TEOS monomer. This gives sol component molar ratios of 1.2 TEOS:42 H₂O:22 EtOH with pH˜5. In addition to the silicate precursor, alcohol, water, which are present with the noted relative molar ratios, the composition would also further include the infusing agent, e.g., Cu(NO₃)₂ or GaCl₃, which can be present in a range of values depending on loading percentage. To prepare fluorine silica sol infused with NH₄F precursor, the above recipe was modified by replacing the DI water (0.75 g) with equal amount of 1 N aqueous HCl solution to reduce pH and slow silica gelation to enable droplet microfluidic emulsification for up to two hours. The most NH₄F possible for the precursor was 5 mg (0.14 mmol); exceeding this leads to rapid gelation of the sol as well as irregularly shaped microspheres. While establishing the source of fluorine infusion, triethoxyfluorosilane (TEFS), and sodium fluoride (NaF) were also incorporated into the sol. TEFS seemed promising for fluorine-loaded silica considering it has structural similarity to TEOS, including a covalently linked fluorine. Both NaF and TEFS displayed fast gelation of the sol precursor even at low concentrations. Therefore, they were not as amenable to processing in a microfluidic device. NH₄F did not display nearly as rapid gelation, but modification to the standard recipe was implemented to remain flowable through a fluidic device. The molar ratio for the NH₄F-infused sol is 1.2 TEOS:41 DI H₂O:22 EtOH:0.00075 HCl with a pH of ˜1. In addition to the silicate precursor, alcohol, water, and acid, which are present with the noted relative molar ratios, the composition would also further include the infusing agent, e.g., Cu(NO₃)₂ or GaCl₃, which can be present in a range of values depending on loading percentage. Stoichiometry of the sol precursors can be altered, to a degree, to influence the percentage of the infusion species. For example, decreasing the infusion precursor leads to a corresponding reduction in final infusion percentage.

### Ga-68 Sol Preparation

GaCl₃-68 was obtained from a germanium-68/gallium-68 generator and came eluted in 0.1M HCl. The maximum Ga-68 concentration available from the generator was-14 mCi per 5 mL (2.8 mCi/mL) solution. At least two half-lives pass during the fabrication process due to microfluidic processing and furnace treatment alone. Therefore, the precursor was adjusted to include at least 10 mCi GaCl₃-68 activity to compensate for radioactive decay during fabrication. Due to the diluted product which is eluted from the germanium-68/gallium-68 generator, an additional concentration step was utilized to have a satisfactory radioactive yield in the final microsphere sample. A simple method of concentrating the generator elute was found in evaporating excess water with a hot plate shielded by lead bricks. The GaCl₃-68 elute was transferred to a 20 mL glass vial and heated at 100° C. to evaporate the 5 mL aqueous solution. The hot plate took 20-30 minutes to dry the 5 mL solution, care was used to avoid overheating the elute given the boiling point of GaCl₃ is 201° C. While some activity was lost to decay time, the hot plate concentration resulted in dry, concentrated GaCl₃-68.

### Droplet Microfluidics

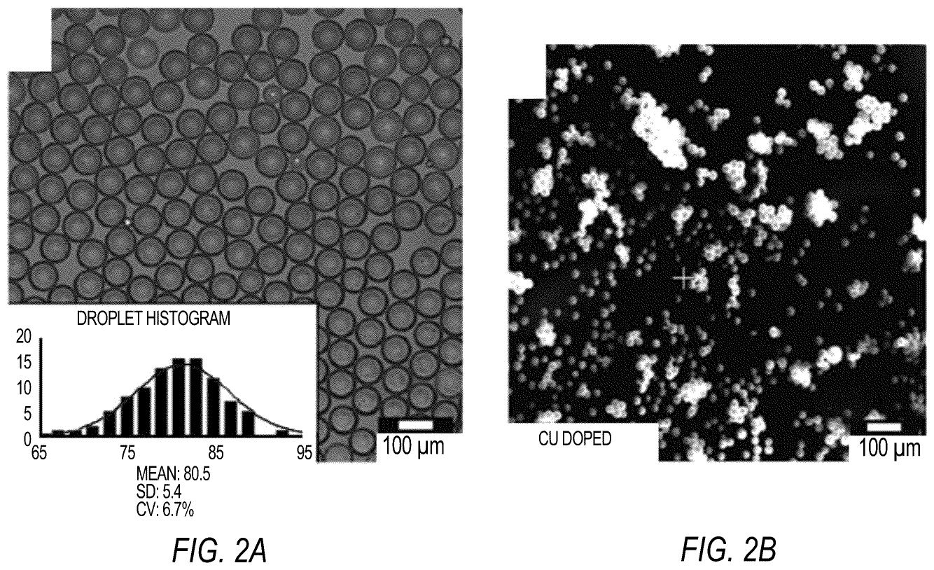

Photoresist-templated (SU-8) poly(dimethylsiloxane) (PDMS) microfluidic devices were fabricated using established soft-lithography methods.⁵⁵ An oil continuous fluid was prepared by dissolving a copolyol surfactant with the trade name ABIL EM 90 available from Degussa (Frankfurt, Germany) in hexadecane (4 wt %). Emulsification of aqueous infused silica precursors was achieved by supplying the aqueous sol phase and organic oil continuous fluid to the microfluidic device using two digitally controlled Harvard PHD 2000 syringe pumps. To achieve 80 μm diameter droplets, the volumetric flow rate for the dispersed sol was 0.25 mL/hr with a flow rate of 0.25-0.5 mL/hr for the continuous oil fluid. The droplet (and therefore particle) production was approximately 500,000-800,000 per hour. FIG. 2A demonstrates a representative collection of monodisperse aqueous sol precursor drops as imaged by brightfield microscopy. The average size of this droplet population was 80.5±5.4 μm with a 6.7% coefficient of variation. The pre-processed microfluidic droplets can have a coefficient of variation of, for example, less than 10%, less then 7%, about 5% to about 7%, about 5% to about 10%, about 1% to about 7%, or about 1% to about 10%.

### Thermal Treatment

The collected microfluidic produced sol droplets were placed into a tube furnace for two distinct heating steps. The first step is at 200° C. for 30 minutes under vacuum to polymerize the droplets by solvent evaporation. This was followed by thermal treatment of the resultant infused silica particles by heating at 400° C. (for fluorine infusion) or 600° C. (for copper and gallium infusions) to consolidate the infusion/silica matrix and evaporate remaining organics, such as the oil phase. Finally, the microspheres were washed with ethanol, centrifuged, and dried.

### Ga-68 Microspheres Saline Rinse

To test stability of the radioactive microspheres, the sample was subjected to ten vigorous rinse cycles with 0.17 M NaCl saline solution. The rinsing is meant to simulate in vivo response of the microspheres and ensure leaching of the Ga-68 radiotracer would not occur. Leaching was determined by measuring the pre-rinse radioactivity with a dose calibrator followed by measuring the post-rinse microspheres and saline supernatant. The microspheres were centrifuged, and the saline supernatant was removed and checked for radioactivity in the dose calibrator.

### SEM and EDS

A SEM equipped with energy dispersive spectroscopy (EDS) was employed for particle imaging and to evaluate morphology and atomic composition of the particle surface (˜2 μm in depth). SEM samples were prepared by mounting microspheres on carbon tape. Samples were analyzed at 15 kV and 5-10 nA. The software used for collection is EDAX Genesis. EDS measurements were carried out in triplicate for each microsphere, and the experimental loading percent results are compared to the ideal case of complete condensation of silica, no loss of infusion material, and complete removal of volatile solvent during thermal treatment. The idealized reaction for silica formation in this case is: Si(OC₂H₅)₄+2H₂O→SiO₂+4C₂H₅OH.

### Electron Probe Microanalysis

Electron probe microanalysis (EPMA) was used to determine the chemical composition of the microsphere bulk by examining cross-sectioned particles. Particles were embedded in epoxy, then ground on 600 grit silicon carbide paper to expose the cross-section of the spheres. Since the cross-section varies on the circumferential point, the microspheres appear polydisperse in these samples. The surface was smoothed with 9, 6, 3, and 1 μm diamond paste on nylon polishing pads and the polished sample was coated with carbon to provide proper surface conductivity for analysis with the electron microprobe. The sample was analyzed using a JEOL 8200 electron microprobe. Analyses were conducted at 15 kV and 5-10 nA with a spot size of 5-10 μm. Oxygen and silicon were analyzed using alumina (0) and quartz (Si) as calibration standards, with 30-second peak counting time. Probe Software's Phi-Rho-Z correction program for EPMA was used to convert the measurements to numerical elemental percent.

### Elemental Analysis

TABLE 1

| | | |
|---|---|---|
| Element | Hypo. Mol % | Exp. Mol % |

Copper

| | | |
|---|---|---|
| Si | 31.9 | 32.4 |
| O | 63.8 | 63.7 |
| Cu | 4.2 | 3.6 ± 0.59 |

Gallium

| | | |
|---|---|---|
| Si | 32.7 | 33.8 |
| O | 65.5 | 63.8 |
| Cl | — | 0.4 |
| Ga | 1.8 | 1.7 ± 0.21 |

Fluorine

| | | |
|---|---|---|
| Si | 32.7 | 34.8 |
| O | 65.4 | 63.8 |
| F | 1.9 | 1.2 ± 0.58 |

Table 1 presents representative EDS elemental mol % characterization of copper, gallium (nonradioactive), and fluorine doped silica particles. Each sample represents the highest loading efficiency achieved for the respective infusing agent. The maximum hypothetical mol % is compared to experimentally measured mol % for each sample.

### Morphology

FIGS. 2B and 2C show SEM images of post-furnace treatment copper-infused silica particles and fluorine-infused silica particles, respectively. The SEM analysis reveals nearly uniform microspheres. The average particle diameter for fluorine-infused particles is 21.4±2.7 μm, 22.5±2.3 μm for gallium-infused (nonradioactive), and 22.3±3.0 μm for copper-infused particles. The main determinant of size was flow rate and channel size. See FIG. 2D for size distribution histograms. Particle size and dispersity is similar regardless of infusing agent. Fluorine-infused sol precursor droplets began to gel before thermal treatment, which impacted the final shape of fluorine-infused microspheres to be less spherical than that of copper and allium-infused (FIG. 2C inset). The sphericities were 0.91 for fluorine-infused compared to 0.98 for copper- and gallium-infused. FIG. 2D shows infused silica glass microspheres having a coefficient of variation ranging from 10.2% to 13.5%. In various further aspects, the infused silica glass microspheres can have a coefficient of variation of less than or about 20%, 15%, or 10%. In various further aspects, the infused silica glass microspheres can have a coefficient of variation of less than or about 10% to about 15%, about 5% to about 15%, or about 1% to about 15%.

### Copper-Infused Silica

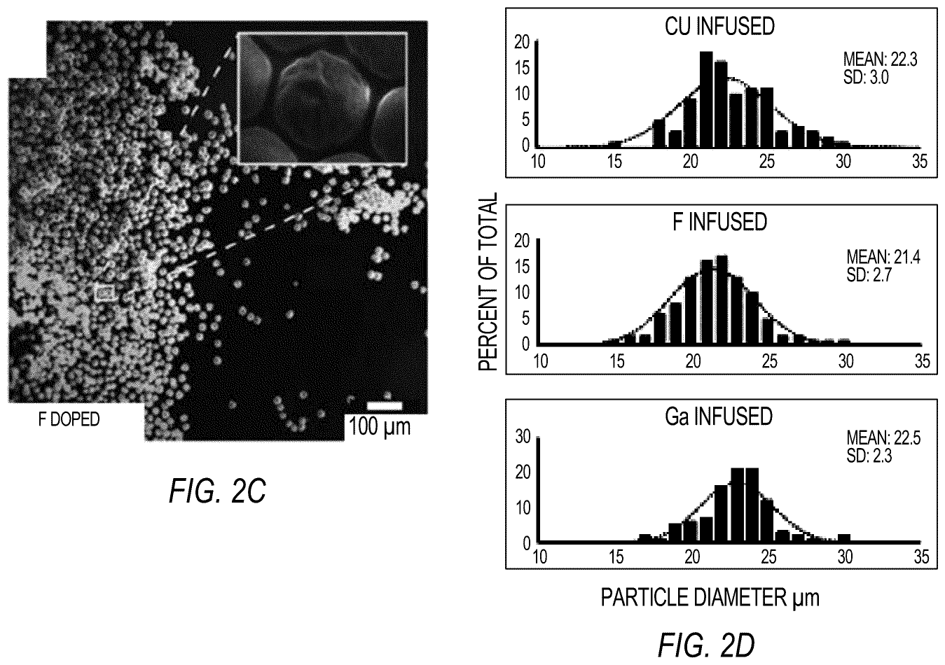

Five distinct samples of copper-infused silica particles with incrementally increasing Cu(NO₃)₂ content in the sol from 0.08 mmol (15 mg) to 0.25 mmol (50 mg) were investigated. Copper loading for each sample was characterized using EDS analysis as shown in FIG. 3A. For all samples, the copper percentage of the silica microspheres increased with increasing copper nitrate added to the sol. However, as the amount of copper nitrate was increased above ˜0.2 mmol, the efficiency of copper loading (fraction of initial copper retained in the particles) decreased. The highest copper loading efficiency (85%) observed used 0.16 mmol copper nitrate. The mol % for Cu, Si, and O in this sample was measured via EDS analysis and are presented in Table 1.

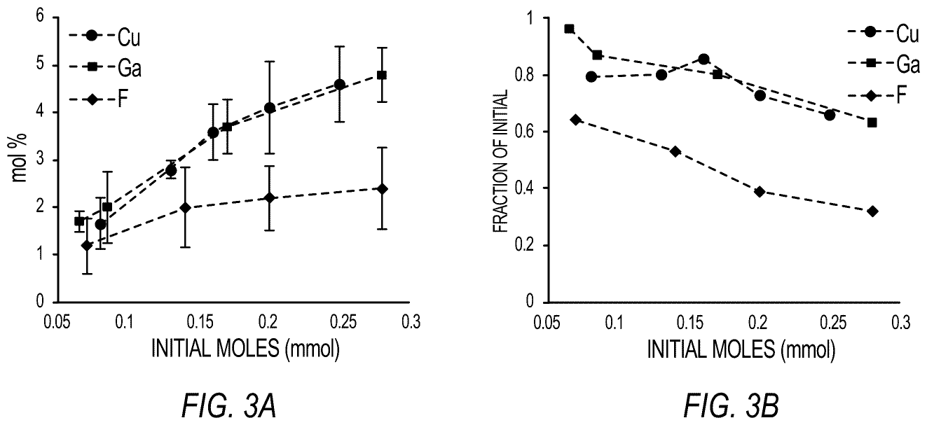

For all copper-infused samples, no elemental nitrogen was detected by EDS in the particles, suggesting volatile nitrogen-based byproducts evaporate completely during furnace treatment. When using 0.16 mmol of copper nitrate in precursor, the copper content can vary between one and five mol % throughout a single microsphere. At this molar ratio, an average of 3.6 mol % (10.6 wt %) copper per microsphere was observed, with large standard deviation (Table 1). The large copper variance across a single particle was subsequently validated by EPMA, which reveals copper oxide microstructures segregating from the bulk silica matrix (FIG. 4). 3.6 mol % indicates some copper loss throughout processing when compared to the maximum hypothetical mass percent of 4.2 mol % at 0.16 mmol. The reduction in loading efficiency with increasing copper precursor might be linked to the observation that, when a sphere exceeds roughly 5 mol % (15 wt %) copper, deposits of copper on the surface of the sphere are seen.

### Fluorine-Infused Silica

Fluorine-infused silica particle samples were synthesized with increasing NH₄F added to the precursor. The amounts ranged from 0.07 mmol (2.5 mg) to 0.28 mmol (10 mg) and determined fluorine loading percent in the microspheres using EDS analysis. As with copper and gallium, the percentage of fluorine in the silica particles increases with increasing NH₄F precursor amount. Unlike copper and gallium, significantly lower loading efficiencies were detected (FIG. 3B). The fluorine loading efficiency of the particles decreased from 64% to 32% with increasing concentration of NH₄F in the precursor. NH₄F reacts to form volatile HF upon addition to the aqueous sol, resulting in significant losses of fluorine during thermal treatment steps. Exposure at 400° C. for 60 minutes in the furnace was found to be a sufficient temperature for stabilizing the silica matrix while preserving most of the fluorine present in the droplets. Higher fluorine was retained at a reduced thermal treatment temperature of 320° C., however, this temperature did not evaporate all organic materials off the sample. Fluorine volatility is based on temperature, given that calcining the fluorine-infused particles at 600° C. resulted in only trace fluorine being detected by EDS.

Fluorine experiments displayed faster silica sol gelation kinetics with increasing amounts of unintentionally catalytic NH₄F. Infused silica samples prepared with 0.28 mmol NH₄F sols comprised up to 2.4 mol % (2.3 wt %) fluorine but after processing in the microfluidic device for around 20 minutes gelation of precursor clogged the device. 0.14 mmol was the largest molar amount of NH₄F allowed in the precursor that did not cause untimely gelation, but this led to low (<2 mol %) fluorine in the microspheres.

### Gallium-Infused Silica

Four distinct nonradioactive gallium-infused silica particle samples were prepared, each distinguished by increasing amounts of precursor GaCl₃, ranging from 0.065 mmol (12 mg) to 0.28 mmol (50 mg). FIG. 3 shows that for all samples, the gallium percent in the final silica microspheres increased with increasing GaCl₃ in the sol. However, the gallium loading efficiency in the silica spheres decreased while increasing molar amount of precursor GaCl₃ added to the sol. For example, the highest amount of GaCl₃ precursor used in this study was 0.28 mmol which led to an average of 4.8 mol % (14.5 wt %) gallium in the particles. The hypothetical gallium content for this sample is 7.65 mol %, so the loading efficiency is ˜63%. The most efficient gallium loading efficiency occurred when using 0.065 mmol, where the average gallium content was 1.7 mol % (5.8 wt %). This is close to the 1.77 mol % hypothetical gallium content, the loading efficiency for these gallium-infused microspheres is 96%. The mol % for Ga, Si, O, and Cl based on EDS analysis is presented in Table 1. Note the presence of trace Cl (0.4 mol %) detected by EDS in this sample. Electron probe microanalysis conducted on this sample also suggests possible trace Cl in the particles. The chlorine presence might be attributed to a partially hydrolyzed gallium framework site as reported by others.

### Microstructure

The differences in microstructure of silica infused with copper and gallium following thermal treatment at 400° C. were investigated using EPMA. The copper microspheres exhibit small, bright copper spheres (3 μm or smaller) within the bulk of the silica matrix. The backscattered electron image is sensitive to atomic weight, so brightness results from greater electron scattering due to the larger nuclei of copper relative to the surrounding silicon and oxygen. These microstructures are consistent with previous findings in which Cu(NO₃)₂ is infused into a silica matrix using sol-gel chemistry. A previous investigation using similar Cu(NO₃)₂-infused sols and processing temperature of 400° C. revealed copper oxide and/or copper as the dispersed copper particle species. Thus, the bright caches are likely a segregation of copper oxide, dispersed within the lower atomic weight silicon and oxygen matrix (FIG. 4A).

In contrast to copper-infused particles, gallium-infused particles showed a homogeneous distribution throughout the particle interior (FIG. 4B). X-ray photoelectron spectroscopy (XPS) characterization done in other studies has shown a similar dispersion of gallium in glass. Studies have shown that Ga³⁺ ions incorporated into the silica framework likely form a tetrahedral bond with OH groups. Based on the apparent homogenous gallium distribution, the availability of a germanium-68/gallium-68 generator, and the observation of high loading efficiency for gallium-infused particles, Ga-68 was the chosen isotope for radioactive microsphere synthesis.

### Ga-68 Silica

When applying this system to produce radioactive microspheres, a total of ˜0.5 mL sol precursor was prepared, which included 10.5 mCi GaCl₃-68. The precursor was processed for one hour and processed 0.25 mL worth of droplets. Due to less than half of the sol volume being processed and a half-life decay of Ga-68, the monodisperse droplet sample contained 1.21 mCi. The droplets were solidified, and the solvents evaporated during the one-hour furnace treatment. After the sample cooled for 10 minutes, the remaining sample activity was 607 μCi compared to an expected 587 μCi based on decay calculations. Hence, the final sample contained 607 μCi distributed among roughly 5×10⁵ microspheres.

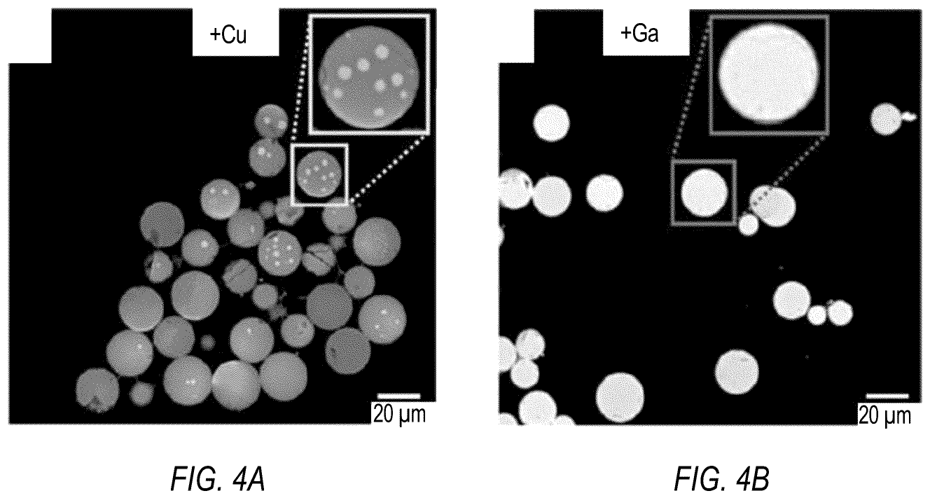

Electron microscopy revealed nearly monodisperse microspheres which were morphologically similar to nonradioactive gallium microspheres (FIG. 5). The mean particle diameter was 23±2.9 μm with >90% of the measured particles between 17 and 27 μm in diameter. EDS measured an average of 33.1 wt % silica and 66.7 wt % oxygen. There was no detectable gallium or zinc (the daughter isotope of gallium-68) since the mass of gallium corresponding to ˜10 mCi in the precursor sol is relatively low.

### Saline Rinses

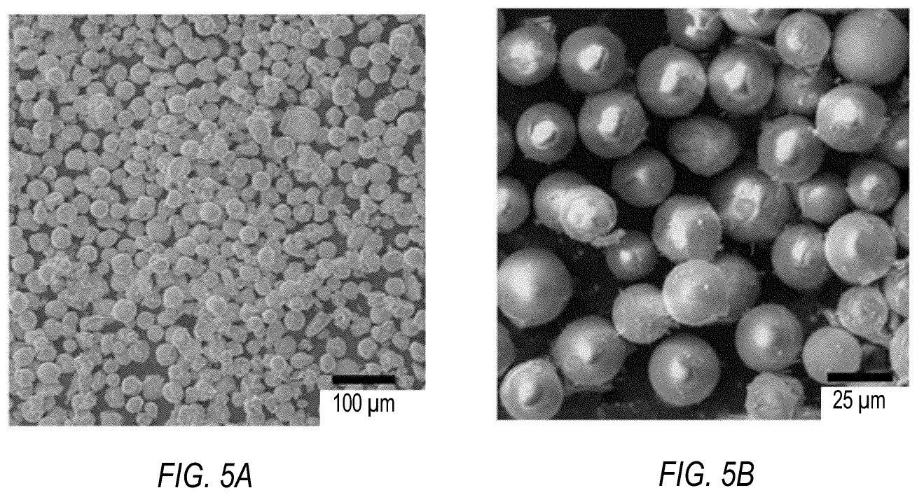

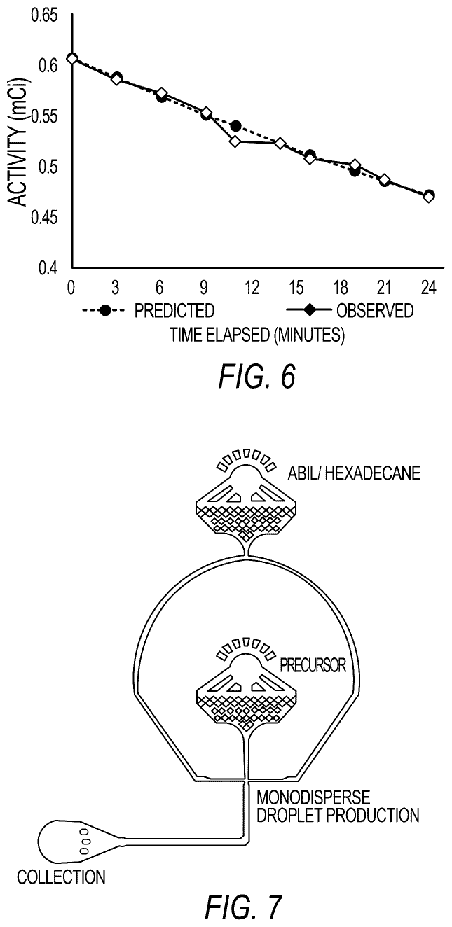

Physiological pH saline rinses were done to assess stability of the infused Ga-68 microspheres. Table 2 shows the dose calibrator measurements of the microspheres and supernatant discard vial between each rinse cycle. FIG. 6 depicts expected activity based on Ga-68 decay plotted alongside the measured sample activity. Saline rinse cycles of Ga-68 microspheres indicated high particle stability, with no activity leaching detected in waste rinse measurements. This result coincides with the high loading efficiency observed in the nonradioactive gallium silica fabrication. Like Y-90 glass, the radioactive agent seems to be stable within the microsphere bulk and is unlikely to leak the radiotracer.

TABLE 2

| Minutes | Predicted Activity | Product Activity | Waste Activity |
|---|---|---|---|
| Elapsed | (mCi) | (mCi) | (mCi) |
| 0 | 0.607 | 0.607 | 0 |
| 3 | 0.588 | 0.586 | 0 |
| 6 | 0.569 | 0.573 | 0.0001 |
| 9 | 0.551 | 0.554 | 0 |
| 11 | 0.54 | 0.525 | 0 |
| 14 | 0.523 | 0.523 | 0.0001 |
| 16 | 0.512 | 0.508 | 0.0001 |
| 19 | 0.496 | 0.502 | 0.0001 |
| 21 | 0.486 | 0.487 | 0.0001 |
| 24 | 0.471 | 0.469 | 0.0001 |

Table 2 shows a table of activity measurements throughout the ten saline rinses. All expected and product activity measurements are within 1% of one another. There was no significant activity detected in the waste for any of the cycles, indicating highly stable particles.

### Radioactive Imaging Microspheres

Valuable information can be obtained for hepatocellular carcinoma from injecting PET imaging microspheres such as tumor-to-liver ratio and lung shunting factor. Ga-68 microspheres closely match between particle density and size distribution of therapeutic Y-90 glass spheres. Using this system of fabrication, radioactive Cu-64 or F-18 microspheres could also be synthesized. Multiple radiotracer options are useful in case different half-lives are desired or due to lack of isotope availability. As mentioned before, F-18 is the most commonly used PET radiotracer and has proven its capabilities as a body-wide diagnostic PET radiotracer when conjugated with deoxyglucose. One concern based on the findings of fluorine halogen infusion was volatility when subjected to high temperatures. Fluorine had lower relative loading efficiency which indicates probable F-18 evaporation during furnace treatment. Copper-infused microspheres, however, showed high loading efficiency, which means they would also likely prove to be a stable PET radiotracer.

### Discussion

In addition to medical imaging and cancer therapeutics, infused silica glass has gained widespread attention for its utility across diverse biomedical applications such as drug delivery, tissue engineering, and as bioactive glasses. For example, bioactive copper-doped glasses display increased osteoblast production, spur angiogenesis, and exhibit antimicrobial effects. Gallium-infused glasses can modulate plasma membrane permeability, treat hypercalcemia, inhibit bone resorption, and have antibacterial activity. The fabrication method described herein could also be adapted to accommodate other elemental infusions besides the three detailed here, especially considering the high loading efficiency of metal infusions. Iron, zinc, silver, calcium, zirconium, and cerium, for example, are other metals that would likely be simple to integrate into the system described here and have shown utility in biomedical implementations.

In conclusion, a benchtop flow-focusing microfluidic system for producing silica microspheres with controllable guest additives such as copper, fluorine, and gallium is introduced. Tailorable infusion composition is attainable with a narrow particle size distribution and demonstrated tunability of particle size as a function of precursor constituent molar ratios, microfluidic flow rates, and channel dimensions of the device chip. Alterations of the sol recipe and microfluidic device features enable personalized, point-of-care treatments by fitting specific patient attributes or goals. The process uses a single-pot synthesis comprising a modified sol precursor infused with copper nitrate, gallium chloride, or ammonium fluoride for monodisperse silica microspheres containing up to 5 mol % copper or gallium, and up to 2 mol % fluorine. This disclosure additionally represents the first fabrication of positron-emitting silica microspheres, the time of fabrication was ˜two hours which is practical when working with rapidly decaying radioisotopes. The Ga-68 particles described replicate the density and size range of commercially available and clinically used glass Y-90 microspheres. Such radiotracers would provide utility as pretreatment for SIRT candidates by leveraging the many advantages of PET imaging and representing the most similar surrogate imaging microspheres to date.

### Exemplified Aspects

The following aspects are provided as example aspects of the various disclosed subject matter:

Aspect 1 provides a composition comprising a monodispersion of silica glass microspheres infused with a positron emission tomography (PET) radiotracer.

Aspect 2 provides composition of Aspect 1, wherein the silica glass microspheres have a diameter of 10 μm to 50 μm.

Aspect 3 provides the composition of any one of Aspects 1-2, wherein the silica glass microspheres have a diameter of 20 μm to 30 μm.

Aspect 4 provides the composition of any one of Aspects 1-3, wherein the radiotracer is a radionuclide.

Aspect 5 provides the composition of any one of Aspects 1-4, wherein the radiotracer is copper, gallium, zinc, fluorine, iron, zinc, silver, calcium, zirconium, or cerium.

Aspect 6 provides the composition of any one of Aspects 1-5, wherein the radiotracer is Cu-64, Ga-68, Zr-89, and F-18.

Aspect 7 provides the composition of any one of Aspects 1-6, wherein the monodispersion is >95% particles having a diameter of 10 μm to 50 μm.

Aspect 8 provides the composition of any one of Aspects 1-7, wherein the monodispersion has a coefficient of variation of less than 15%.

Aspect 9 provides the composition of any one of Aspects 1-8, which is formulated for use as a pre-treatment radiotracer to predict distribution of radioactive Y-90 microspheres.

Aspect 10 provides the composition of any one of Aspects 1-9, comprising a pharmaceutically acceptable carrier.

Aspect 11 provides a method of preparing a monodispersion of silica glass microspheres infused with a positron emission tomography (PET) radiotracer, comprising:
preparing an aqueous sol of infused silica precursors from a silicate precursor, a radiotracer precursor, and an aqueous solvent;
preparing microfluidic droplets from an emulsification of the aqueous sol and an organic oil continuous fluid dispersed by a microfluidic device; and
providing a two-step thermal treatment, which includes a first thermal step that heats the microfluidic droplets sufficiently to polymerize the droplets by solvent evaporation, and a second thermal step that heats the polymerized droplets sufficiently to consolidate an infusion-silica matrix and evaporate residual components.

Aspect 12 provides the method of Aspect 11, wherein the silicate precursor is an alkyl silicate.

Aspect 13 provides the method of any one of Aspects 11-12, wherein the silicate precursor is a tetraalkyl orthosilicate in an alcohol solvent.

Aspect 14 provides the method of any one of Aspects 11-13, wherein the radiotracer precursor is a salt.

Aspect 15 provides the method of any one of Aspects 11-14, wherein the radiotracer precursor is NH4F, GaCl3, or Cu(NO3)2 in water.

Aspect 16 provides the method of any one of Aspects 11-15, wherein the organic oil continuous fluid comprises a C10-C20 hydrocarbon solvent.

Aspect 17 provides the method of any one of Aspects 11-16, wherein the organic oil continuous fluid comprises a copolyol surfactant.

Aspect 18 provides the method of any one of Aspects 11-17, wherein the organic oil continuous fluid comprises hexadecane.

Aspect 19 provides the method of any one of Aspects 11-18, wherein the microfluidic droplets have an average particle size of 60 μm to 100 μm.

Aspect 20 provides the method of any one of Aspects 11-19, wherein the microfluidic droplets have an average particle size of 75 μm to 85 μm.

Aspect 21 provides the method of any one of Aspects 11-20, wherein the microfluidic droplets have a coefficient of variation of less than 10%.

Aspect 22 provides the method of any one of Aspects 11-21, wherein the microfluidic droplets have a coefficient of variation of about 5% to about 7%.

Aspect 23 provides the method of any one of Aspects 11-22, wherein the first thermal step heats the microfluidic droplets at a temperature of 100° C. to 300° C. for 15 minutes to 5 hours.

Aspect 24 provides the method of any one of Aspects 11-23, wherein the first thermal step heats the microfluidic droplets at a temperature of 180° C. to 220° C. for 20 minutes to 1 hour.

Aspect 25 provides the method of any one of Aspects 11-24, wherein the second thermal step heats the microfluidic droplets at a temperature of 300° C. to 900° C. for 5 minutes to 5 hour.

Aspect 26 provides the method of any one of Aspects 11-25, wherein the second thermal step heats the microfluidic droplets at a temperature of 350° C. to 650° C. for 30 minutes to 90 minutes.

Aspect 27 provides the method of any one of Aspects 11-26, which is a one-pot process.

Aspect 28 provides the method of any one of Aspects 11-27, wherein the silica glass microspheres have a diameter of 10 μm to 50 μm.

Aspect 29 provides the method of any one of Aspects 11-28, wherein the silica glass microspheres have a diameter of 20 μm to 30 μm.

Aspect 30 provides the method of any one of Aspects 11-29, wherein the radiotracer is a radionuclide.

Aspect 31 provides the method of any one of Aspects 11-30, wherein the radiotracer is copper, gallium, zinc, fluorine, iron, zinc, silver, calcium, zirconium, or cerium.

Aspect 32 provides the method of any one of Aspects 11-31, wherein the radiotracer is Cu-64, Ga-68, Zr-89, and F-18.

Aspect 33 provides the method of any one of Aspects 11-32, wherein the monodispersion is >95% particles having a diameter of 10 μm to 50 μm.

Aspect 34 provides the method of any one of Aspects 11-33, wherein the monodispersion has a coefficient of variation of less than 10%.

Aspect 35 provides a method of predicting in vivo behavior of therapeutic Y-90 microspheres comprising administering to subject a composition comprising a monodispersion of silica glass microspheres infused with a radiotracer.

Aspect 36 provides a composition comprising a monodispersion of silica glass microspheres infused with a therapeutic nuclide selected from copper, gallium, zinc, fluorine, iron, zinc, silver, calcium, zirconium, and cerium.

Aspect 37 provides the composition of Aspect 36, wherein the therapeutic nuclide is a radionuclide.

Aspect 38 provides the composition of Aspect 36, wherein the therapeutic nuclide is a non-radiologic nuclide.

Aspect 39 provides the composition of Aspect 36, wherein the therapeutic nuclide is biologically active.

Aspect 40 provides a method of preparing a monodispersion of silica glass microspheres infused with a therapeutic nuclide, comprising:
preparing an aqueous sol of infused silica precursors from a silicate precursor, a therapeutic nuclide precursor, and an aqueous solvent;
preparing microfluidic droplets from an emulsification of the aqueous sol and an organic oil continuous fluid dispersed by a microfluidic device; and providing a two-step thermal treatment, which includes a first thermal step that heats the microfluidic droplets sufficiently to polymerize the droplets by solvent evaporation, and a second thermal step that heats the polymerized droplets sufficiently to consolidate an infusion-silica matrix and evaporate residual components.

Aspect 41 provides a kit for fabricating a monodispersion of silica glass microspheres infused with a positron emission tomography (PET) radiotracer, comprising one or more of: a two-channel droplet-based microfluidic device, a silicate precursor, a positron emission tomography (PET) radiotracer precursor, instructions for preparing an aqueous sol of infused silica precursors, instructions for preparing an aqueous sol of infused silica precursors, instructions for preparing microfluidic droplets from an emulsification of the aqueous sol, instructions for providing a two-step thermal treatment for microfluidic droplets to prepare the monodispersion of silica glass microspheres.

Aspect 42 provides a two-channel droplet-based microfluidic device configured for point-of-care fabrication of a monodispersion of silica glass microspheres infused with a positron emission tomography (PET) radiotracer, a therapeutic nuclide, or both.

Aspect 43 provides a system comprising a two-channel droplet-based microfluidic device and a controller configured for automating fabrication of a monodispersion of silica glass microspheres infused with a positron emission tomography (PET) radiotracer, a therapeutic nuclide, or both.

Aspect 44 provides a system, method, or apparatus having any combination or permutation of features recited in one or more of the aforementioned Aspects.

What is claimed is:

1. A composition comprising a monodispersion of silica glass microspheres infused with a positron emission tomography (PET) radiotracer.

2. The composition of claim 1, wherein the silica glass microspheres have a diameter of 10 μm to 50 μm.

3. The composition of claim 1, wherein the radiotracer is a radionuclide.

4. The composition of claim 1, wherein the radiotracer is copper, gallium, zinc, fluorine, iron, zinc, silver, calcium, zirconium, or cerium.

5. The composition of claim 1, wherein the radiotracer is Cu-64, Ga-68, Zr-89, and F-18.

6. The composition of claim 1, wherein the monodispersion is >95% particles having a diameter of 10 μm to 50 μm.

7. The composition of claim 1, wherein the monodispersion has a coefficient of variation of less than 15%.

8. The composition of claim 1, which is formulated for use as a pre-treatment radiotracer to predict distribution of radioactive Y-90 microspheres.

9. The composition of claim 1, comprising a pharmaceutically acceptable carrier.

10. A method of preparing a monodispersion of silica glass microspheres infused with a positron emission tomography (PET) radiotracer, comprising:
preparing an aqueous sol of infused silica precursors from a silicate precursor, a radiotracer precursor, and an aqueous solvent;
preparing microfluidic droplets from an emulsification of the aqueous sol and an organic oil continuous fluid dispersed by a microfluidic device; and
providing a two-step thermal treatment, which includes a first thermal step that heats the microfluidic droplets sufficiently to polymerize the droplets by solvent evaporation, and a second thermal step that heats the polymerized droplets sufficiently to consolidate an infusion-silica matrix and evaporate residual components.

11. The method of claim 10, wherein the silicate precursor is an alkyl silicate.

12. The method of claim 10, wherein the radiotracer precursor is NH₄F, GaCl₃, or Cu(NO3)2 in water.

13. The method of claim 10, wherein the organic oil continuous fluid comprises a C10-C20 hydrocarbon solvent, a copolyol surfactant, or both.

14. The method of claim 10, wherein the microfluidic droplets have an average particle size of 60 μm to 100 μm.

15. The method of claim 10, wherein the microfluidic droplets have a coefficient of variation of about 5% to about 7%.

16. The method of claim 10, wherein the first thermal step heats the microfluidic droplets at a temperature of 100° C. to 300° C. for 15 minutes to 5 hours.

17. The method of claim 10, wherein the second thermal step heats the microfluidic droplets at a temperature of 300° C. to 900° C. for 5 minutes to 5 hour.

18. The method of claim 10, which is a one-pot process.

19. The method of claim 10, wherein the radiotracer is copper, gallium, zinc, fluorine, iron, zinc, silver, calcium, zirconium, or cerium.

20. A method of predicting in vivo behavior of therapeutic Y-90 microspheres comprising administering to subject a composition comprising a monodispersion of silica glass microspheres infused with a radiotracer.
